(19) United States
(12) Patent Application Publication
(10) Pub. No.: US 2005/0096967 A1
(43) Pub. Date: May 5, 2005

(54) Method and apparatus for processing of purchase orders

(76) Inventors: Kevin G. Gerrits, Denver, CO (US); William Jeffrey Oleson, Denver, CO (US); Kevin Ruffe, Superior, CO (US)

Correspondence Address: SIDLEY AUSTIN BROWN & WOOD LLP, 717 NORTH HARWOOD, SUITE 3400, DALLAS, TX, 75201, US

(21) Appl. No.: 10/699,225

(22) Filed: Oct. 31, 2003

(51) Int. Cl. G06F 17/60

(52) U.S. Cl. 705/10; 705/16

(57) ABSTRACT

A method and apparatus for validating, correcting and reconciling purchase orders. In one embodiment, each line item of the purchase order is validated to ensure that it corresponds to an item offered by the supplier. Each line item can be compared to known errors and the erroneous part number replaced with a correct part number. Line items can be modified in accordance with a set of rules. The supplier can be notified of an invalid line item in a purchase order and can correct the invalid line item either before or after the purchase order is received by the supplier. Corrections performed by the supplier can be used to add to the set of rules to be used to process succeeding purchase orders. An electronic catalog of the buyer can be updated after buyer review to correct erroneous part numbers and other related information from various sources.

### FIELD OF THE INVENTION

The invention relates generally to the processing of purchase orders and, more particularly, to a method and apparatus for validating, correcting and reconciling purchase orders.

### BACKGROUND OF THE INVENTION

Generally, a buyer places an order with a supplier for specific items. The buyer completes (i.e., creates or fills out) a purchase order, which includes one or more line items. Each line item generally contains a part number of the item (or other unique identifier), as well as other related information, such as unit of measure, quantity of eaches, quantity, color, size, and the like.

The burden of creating and processing purchase orders can be enormous, particularly in large organizations, such as hospitals, manufacturing plants, retailers, and the like. The purchase order needs to be completed in such a manner that the supplier can correlate a specific product to each line item. Frequently, this entails looking up an item in a parts catalog of a particular supplier and entering the relevant information (e.g., the part number) on the purchase order. Upon receipt of the purchase order, the supplier must examine each line item and correct any inaccuracies before the purchase order can be correctly fulfilled. These tasks are error prone and time consuming.

Computer systems can be used in completing and processing purchase orders. A buyer may maintain a computer system which contains information regarding items that it purchases from one or more suppliers. To create a purchase order, the buyer can indicate the items that it wants to purchase, and the computer system can then generate an appropriate purchase order. The purchase order can then be transmitted to the supplier, via facsimile or other electronic or manual means. Upon receipt of the purchase order, the supplier can enter the information from the purchase order into the supplier's computer system, either electronically or manually. The supplier's computer system can identify inaccuracies in the line items, which must then be corrected before the purchase order can be fulfilled.

The computer systems of the buyer and the supplier are independent and do not interact with each other. As a result, the computer systems do not provide for automatic validation and reconciliation. Rather, errors are generally repeated, even between the same buyer and supplier and with the same information (i.e., the buyer can repeatedly order an item using the same erroneous part identifier).

Therefore, there is a need for a method and an apparatus capable of validating, correcting and reconciling purchase orders. Furthermore, there is a need for a method and apparatus capable of automatically updating rules that govern validating, correcting and reconciling of purchase orders.

### SUMMARY OF THE INVENTION

One aspect of the present invention is directed to a method and apparatus for validating purchase orders. A purchase order, containing at least one line item is received. Each line item includes, among other things, a supplier part number that uniquely identifies an item offered by a specific supplier. Each line item is validated against an electronic catalog to ensure that the part number corresponds to an item offered by the supplier. Once validated, the processed purchase order can then be transmitted to the supplier. Each line item of a purchase order can also be compared to known errors. If a part number in the line item corresponds to one of the known errors, then the erroneous part number can be replaced with a correct part number. Line items can also be modified in accordance with a set of rules, such that the line item(s) conform to a format acceptable to a particular supplier.

A supplier can be notified, in real time, of an invalid line item in a purchase order, such that the supplier can correct the invalid line item. Any changes made can be stored or remembered and applied to future purchase order submissions.

Another aspect of the present invention is directed to a method and apparatus for comparing a purchase order acknowledgement from the supplier to the original purchase order presented by the buyer. Any changes to a line item can be added to the predetermined set of rules to be used when processing succeeding purchase orders.

Another aspect of the present invention is directed to a method and apparatus for updating an electronic catalog of the buyer to correct erroneous part numbers and other related information contained therein.

### BRIEF DESCRIPTION OF THE DRAWINGS

For a more complete understanding of the present invention, and the advantages thereof, reference is now made to the following descriptions taken in conjunction with the accompanying drawings. Throughout the accompanying drawings, like parts are designated by like reference numbers and in which:

FIG. 1 schematically depicts a high level network environment of one embodiment of the present invention;

FIG. 2 is a block diagram illustrating a portion of the operating modules of a computer system for processing purchase orders in accordance with one embodiment of the present invention;

FIG. 3 is a flow diagram depicting a process, performed by a computer system, for validating, correcting and reconciling purchase orders in accordance with one embodiment of the present invention;

FIG. 4 is a flow diagram depicting a process, performed by a computer system, for further processing of purchase orders in accordance with one embodiment of the present invention;

FIG. 5 is a flow diagram depicting a process, performed by a computer system, for processing of purchase orders in accordance with one embodiment of the present invention;

FIG. 6 illustrates an example of an IME interface screen in accordance with one embodiment of the present invention;

FIG. 7 illustrates an example of a Refinery interface screen in accordance with one embodiment of the present invention; and

FIG. 8 illustrates an example of an interface screen that enables a supplier to correct invalid line items in a purchase order in accordance with one embodiment of the present invention.

### DETAILED DESCRIPTION OF THE PREFERRED EMBODIMENTS

In the following discussion, numerous specific details are set forth to provide a thorough understanding of the present invention. However, it will be obvious to those skilled in the art that the present invention may be practiced without such specific details. Well-known elements have been illustrated in schematic or block diagram form in order not to obscure the present invention in unnecessary detail. Additionally, details concerning computer networking, software programming, telecommunications, and the like have been omitted since such details are not necessary to obtain a complete understanding of the present invention, and are within the skills of persons of ordinary skill in the relevant arts.

Unless otherwise indicated, all functions described herein may be performed in either hardware or software, or some combination thereof. For example, in one embodiment, the functions can be performed by a processor such as a computer or an electronic data processor in accordance with code such as computer program code, software, and/or integrated circuits that are coded to perform such functions.

Furthermore, examples of an embodiment of the present invention are described in the context of a medical supply ordering system for illustrative purposes only. The present invention may also be utilized in many different systems and industries, such as restaurant supply ordering systems, automotive parts ordering systems, inventory control systems, and the like.

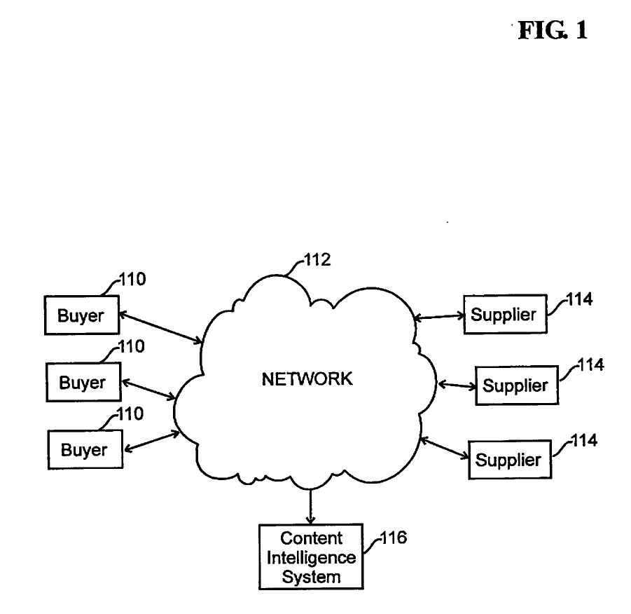

FIG. 1 schematically depicts a high level network environment of an embodiment of the present invention. A network 112 provides a communications medium that connects buyers 110 and suppliers (e.g., manufacturers or distributors) 114 to a content intelligence system (CIS) 116.

The buyer 110, supplier 114 and CIS 116 may include computer systems, such as a mainframe, mini-computer, workstation, personal computer, cellular telephone, personal digital assistant (PDA), or the like, which may be configured to provide a textual (alphanumeric) and/or graphical display (e.g., to a user) and to receive data (e.g., from a user). The computer systems of the buyer 110, supplier 114 and CIS 116 may be coupled to the network 112 via a wired or wireless communication system or any combination thereof. The network 112 can be a private network, a public network, or any combination thereof, including the Internet, a local-area network (LAN), a wide-area network (WAN), or the like.

The CIS 116 is designed to validate, correct, and reconcile purchase orders, thereby increasing the accuracy of transactions between buyers 110 and suppliers 114. The computer system(s) of the buyer 110 may include a legacy materials management information system (MMIS) which maintains a copy of product and pricing information regarding products offered by one or more suppliers 114 and used by the buyer 110. The CIS 116 advantageously allows the buyer 110 to benefit from correct purchase orders without requiring that the buyer 110 synchronize or update all of its existing MMIS data.

In one embodiment, the CIS 116 is a stand-alone computer system configured for receiving a purchase order (e.g., from the buyer 110) and transmitting the purchase order (e.g., after processing) to the supplier 114. The CIS 116 is also configured for receiving a purchase order acknowledgement from the supplier 114 and transmitting the purchase order acknowledgement back to the buyer 110. In another embodiment, the CIS 116 operates as a component of an electronic exchange. The exchange can include, among other things, computer and computer network related devices for receiving and transmitting purchase orders between buyers 110, sellers 114 and the CIS 116. The exchange can, for example, convert purchase orders received from a buyer 110 in a first format to a format compatible with the CIS 116. In alternative embodiments, the functions provided by the CIS 116 may be distributed and may be implemented within the computer system of one or more suppliers 114, a computer system of one or more buyers 110, or any combination thereof. In such an embodiment, the computer system of a buyer 110 may be configured to directly communicate with the computer system of the selected supplier 114.

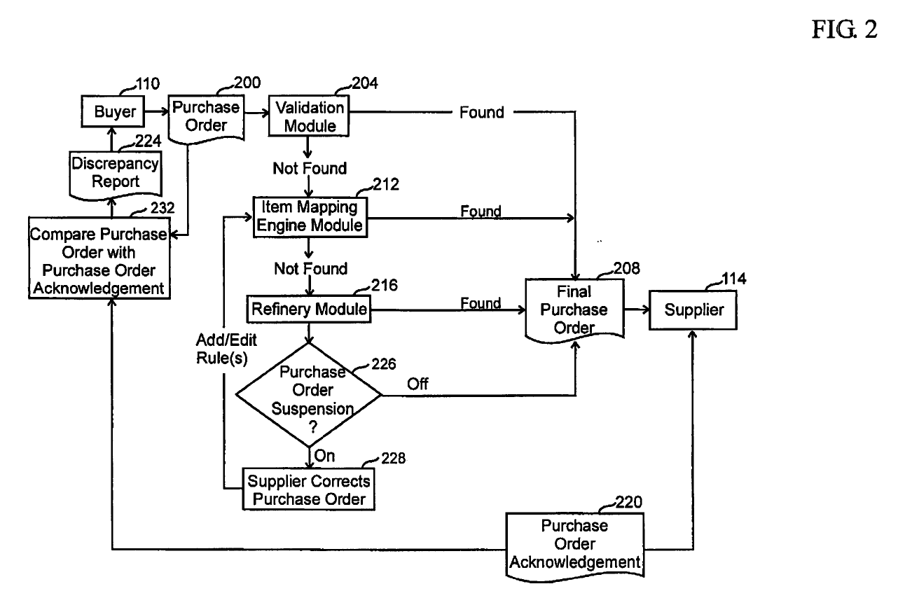

FIG. 2 is a block diagram illustrating a portion of the operating modules of a computer system (e.g., CIS 116) for processing purchase orders 200 in accordance with one embodiment of the present invention. The computer system receives a purchase order 200 from a buyer 110 and processes it before transmitting it to a supplier 114.

The computer system includes a Validation Module 204 for validating the purchase order 200 (which may be directed to a specific supplier 114). The Validation Module 204 looks up each line item (e.g., in an electronic catalog) and determines whether the relevant information (e.g., supplier part number) contained in each line item corresponds to an item offered by the supplier 114. If the line item is found, the line item is valid and can be added to a final purchase order 208. If the line item is not found (e.g., the purchase order contains an incorrect supplier part number as a result of outdated or incorrect information), the computer system can attempt to correct the invalid line item. A line item may contain both the buyer's internal information (e.g., part number, unit of measure, etc.) and the supplier's information (e.g., supplier part number, supplier unit of measure, etc.). In one embodiment, an electronic catalog exists for each trading partner relationship and the Validation Module 204 can validate the line items to the relevant electronic catalog based on the particular buyer 110 and supplier 114. In another embodiment, the Validation Module 204 can validate the line items to a comprehensive electronic catalog containing information relevant to all buyers 110 and suppliers 114.

The computer system optionally includes an Item Mapping Engine Module 212 for correcting errors by comparing the line item to a list of known errors. In one embodiment, a list of known errors is provided for each trading partner relationship. As used herein, a trading partner relationship refers to a relationship between a particular supplier 114 and a particular buyer 110 (or group of buyers). Optionally, the list of errors may be implemented for all buyer organizations. If, for example, a part number in the line item corresponds to one of the known errors, then the erroneous part number can be replaced with a correct part number. In one embodiment, supplier information (e.g., supplier part number) is validated, while the buyer information (e.g., buyer's internal part number or reference) is neither validated nor changed. Once corrected, the line item can be re-validated by the Validation Module 204 to ensure that the application of the rule from the Item Mapping Engine Module 212 resulted in a valid item. The corrected line item can then be added to the final purchase order 208.

The computer system optionally includes a Refinery Module 216 for correcting errors with reference to a set of rules. In one embodiment, a set of rules is provided for each trading partner relationship. Optionally, the set of rules may be implemented for all buyer organizations. By modifying the line item(s) in the purchase order in accordance with the set of rules, the line item(s) can be made to conform to a format acceptable to the particular supplier 114. The line item can then be re-validated by the Validation Module 204 to ensure that the application of the rule (or set of rules) resulted in a valid item. The corrected line item can then be added to the final purchase order 208.

In one embodiment, if errors are detected (e.g., by the validation process), the computer system can notify the supplier 114 in real time (e.g., via email, XML or EDI). The supplier 114 then has the option to correct the invalid line item entry before the purchase order is sent to the supplier 114. If the supplier 114 chooses this approach (purchase order suspension is ON in step 226), the purchase order is suspended and the supplier 114 is notified. The supplier 114 can then correct any invalid line items (in step 228). The corrections performed by the supplier 114 can be used to add new rules or edit existing rules in the Item Mapping Engine Module 212 for correcting the same errors in future purchase orders to the same supplier 114. If the supplier 114 does not choose to correct the invalid line items before the purchase order is sent to the supplier 114 (purchase order suspension is OFF in step 226), the supplier 114 can perform the corrections after it receives the final purchase order 208.

In one embodiment, wherein the supplier 114 chooses to correct the purchase order after it has been received by the supplier 114, the computer system includes a module 232 for comparing a purchase order acknowledgment 220 received from the supplier 114 to the original purchase order presented by the buyer 110. Changes to a line item (e.g., made by the supplier 114, the Item Mapping Engine Module 212, the Refinery Module 216) can be used to create a discrepancy report 224, which can be transmitted (e.g., electronically) back to the buyer 110. In one embodiment, the buyer 110 can receive the discrepancy report 224 (e.g., in text form) and, optionally, the purchase order acknowledgement 220 via XML or EDI. Alternatively, the discrepancy report 224 (and, optionally, the purchase order acknowledgement 220) may be transmitted to the buyer 110 via other electronic or manual methods, such as facsimile, mail, courier, voice, and the like. In another embodiment, discrepancy reports 224 can be aggregated and made available to the relevant buyers 110 (e.g., via a download from the Internet). In such an embodiment, the buyer 110 can enter the CIS 116 (e.g., via a web interface) and access the discrepancy report(s).

In one embodiment, the buyer 110 can use the discrepancy report 224 to update its MMIS (or electronic catalog) to reflect the current product information as specified by the supplier 114. By using the discrepancy report 224, the buyer 110 can update the MMIS based on its recent transactions. Thus, product data that is not used (and that are contained in the MMIS) need not be updated. In one embodiment, the buyer 110 can specify a file format or type (e.g., Excel spreadsheet, ASCII file, etc.) for the discrepancy report 224, such that the report can be loaded into the MMIS. In a related embodiment, the buyer 110 can select certain items in the discrepancy report 224 and specify a file format for a file containing only the selected items. The buyer 110 can thus load into the MMIS only those selected items. In one embodiment, the discrepancy report 224 can be formatted such that the computer system of the buyer 110 can utilize the information in the discrepancy report 224 to update the Item Mapping Engine module 212 for correcting the same errors in future purchase orders to the same supplier 114.

In another embodiment, the buyer 110 can be presented with a interface (e.g., a GUI) for reviewing discrepancies from one or more purchase orders. For example, the buyer 110 may be presented with a list of discrepancies for all purchase orders in the past month. The buyer 110 can review each discrepancy (e.g., both the submitted information and the corrected information) and, for each discrepancy, select whether to include the corrected information in a file for updating the buyer's MMIS or electronic catalog. The discrepancies may include discrepancies in part number, pricing, or other information. In yet another embodiment, the buyer 110 can use the interface (e.g., a GUI) to select from a number of sources of information for updating the buyer's electronic catalog. The buyer 110 can review items in the source of information and select the whether to include each item in a file for updating the buyer's MMIS or electronic catalog. The buyer 110 can also be given the option to select a file format for a file containing the selected items, such that the file will be compatible with and can be uploaded into the buyer's system, either manually by the buyer, or automatically by the CIS 116.

In one embodiment, the information is discrepancy information created by comparing one or more purchase orders from the buyer 110 to corresponding purchase order acknowledgements from the supplier 114. In another embodiment, the information is discrepancy information created by comparing information in the buyer's electronic catalog with information in a supplier's electronic catalog to determine any differences between the buyer's information and the supplier's information. In yet another embodiment, the information is pricing information from a catalog associated with the supplier 114. The information can also be pricing information from a contract agreement associated with the supplier 114 or a third party, such as a group purchasing agent or other third party pricing negotiator or service.

Thus, the computer system is capable of translating outdated or incorrect buyer information (e.g., part number(s) used by the buyer 110) into correct supplier information (e.g., part number(s) used by the particular supplier 114) and thereby route clean transactions to the suppliers 114.

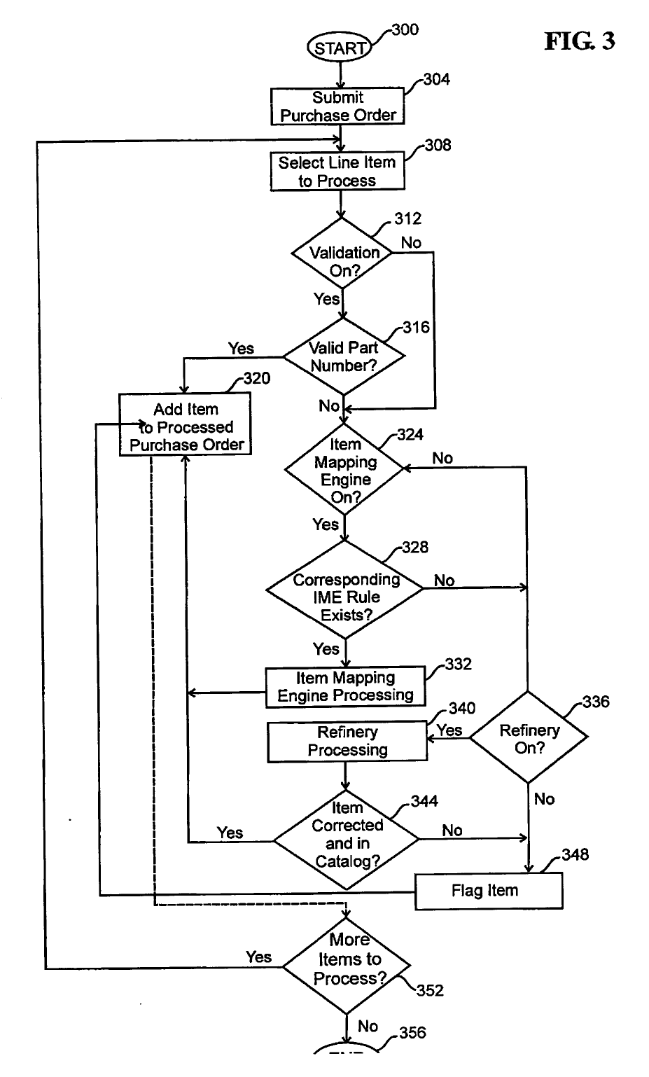

FIG. 3 is a flow diagram depicting a process, performed by a computer system, for validating, correcting and reconciling purchase orders in accordance with one embodiment of the present invention.

The process starts at step 300. In step 304, the buyer 110 submits a purchase order to the CIS 116. The purchase order includes at least one line item, identified by a part number, and may include an indication of the supplier 114 from which each line item is to be purchased. In one embodiment, every line item on the purchase order is to be purchased from a single supplier 114.

In another embodiment, the purchase order may contain a plurality of line items, along with an indication of a specific supplier 114 for each line item or group of line items. The supplier 114 from which each line item is to be purchased may be indicated by a separate field or may be encoded into the part number. The CIS 116 can create a separate purchase order for each supplier 114 based on the original purchase order submitted by the buyer 114. In this manner, each supplier 114 can receive a purchase order containing only the relevant line items, and is not provided information regarding the items purchased by the buyer 110 from another supplier 114.

In step 308, a line item is selected for processing. As discussed above, a purchase order may contain one or more line items. In one embodiment, the line items may be processed in any order (e.g., the order in which the line items appear on the purchase order). Alternatively, some database systems may be able to more efficiently process in the line items in a specific order, such as alphabetically, numerically, ascending or descending key order, or the like.

In a decisional step 312 the CIS 116 determines whether initial Validation is ON for the particular supplier 114 to which the purchase order is directed (e.g., the supplier has opted to provide the CIS 116 with a catalog listing of valid part numbers). If validation is OFF (No in step 312, the process proceeds to step 324). If validation is ON (Yes in step 312), the process continues to step 316.

Then, in step 316, the CIS 116 validates each line item on the purchase order. The CIS 116 maintains a list of known items offered by the supplier 114 in, for example, an electronic catalog. Thus, for each line item, the CIS 116 can verify, for example, that the part number is a valid part number (for the specified supplier 114) by comparing the part number of the selected line item to the part numbers in the catalog. In one embodiment, the CIS 116 is capable of validating one or more of the following fields: (1) Supplier Division, (2) Part Number, (3) Part Number Type, (4) Unit of Measure (UOM), (5) Quantity of Eaches (QOE), (6) Item Status (e.g., whether the item is in production, staging, or on hold), and (7) Part Number Status (e.g., whether the item is orderable).

If the line item is valid (e.g., the part number is in the catalog) (Yes in step 316), then the validation process passes and the line item can be added to a processed purchase order in step 320. The processed purchase order is a purchase order that has been processed by the CIS 116 prior to being transmitted to the supplier 114. If, however, the line item is not valid (e.g., the part number of the selected line item is not located) (No in step 316), then the validation process fails and the process proceeds to step 324.

In one embodiment, the CIS 116 performs validation based on trading partner relationships. For example, it may be desirable for a supplier 114 to establish different part numbers for use by different buyers 110 for the same item (e.g., to track sales to a particular buyer or group of buyers or for custom items). In such an embodiment, the CIS 116 can advantageously verify not only that the part number is valid for the particular supplier 114, but also that the part number is valid for the particular buyer 110, when purchasing from the particular supplier 114.

In step 324, the CIS 116 determines whether Item Mapping Engine (IME) processing is ON for the trading partner relationship. If IME processing is OFF (No in step 324), the process proceeds to step 336. If IME processing is ON (Yes in step 324), the process continues to step 328.

In step 328, the CIS 116 determines whether an IME rule exists that corresponds to the line item. If so (Yes in step 328), the IME rule can be applied (step 332) and the resulting line item added to the processed purchase order in step 320. In one embodiment, the resulting line item (after application of the IME rule) is re-validated (e.g., in step 316) to ensure that the application of the rule resulted in a valid line item. If a corresponding IME rule does not exist (No in step 328), the process proceeds to step 336.

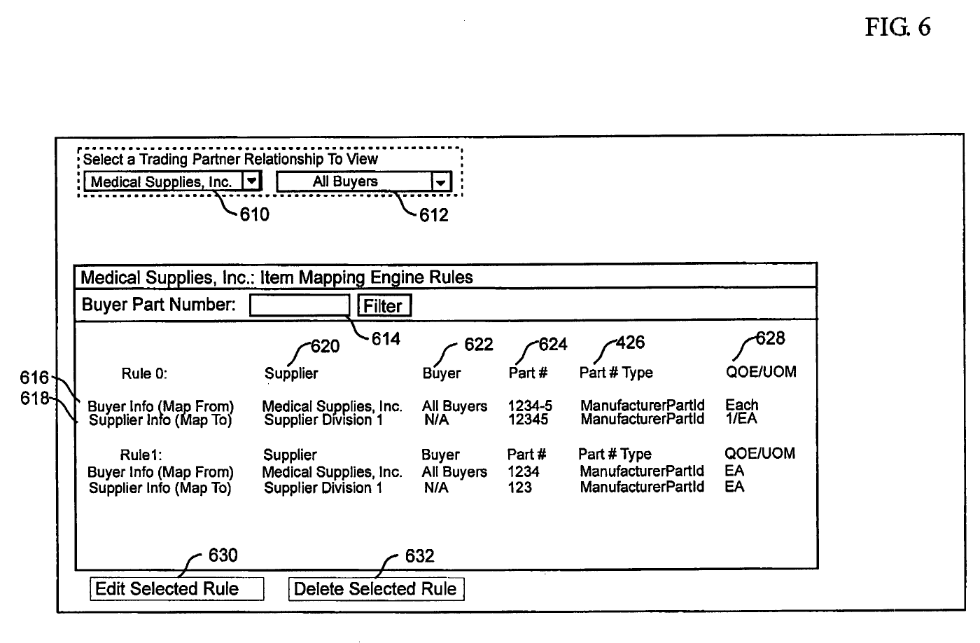

In step 332, the line item is processed by the Item Mapping Engine. The IME is capable of intelligently cross-referencing line item information by using defined translations between a buyer's line item (e.g., “123A45 Case”) and an item in a supplier's catalog (e.g., “12345 CA”) during a transaction. The IME maintains a list of IME rules that identify known discrepancies between the identification of selected line items by buyers 110 and suppliers 114. For example, if a particular buyer 110 is known to incorrectly identify a selected product by the part number “A-123” an IME rule may be created to modify “A-123” to the correct part number: “A-123×.” Each IME rule can be set to apply only to a particular buyer 110, a particular supplier 114, or a particular trading partner relationship. Alternatively, an IME rule can be set to apply to all buyers 110 associated with a supplier 114. The IME rules can be automatically created when a supplier 114 corrects a part number of a selected line item (e.g., as discussed above with respect to steps 226 and 228 of FIG. 2), or can simply be input whenever an error in a part number is detected. An example of an IME interface screen in accordance with one embodiment of the present invention is described below with reference to FIG. 6.

In one embodiment, an IME rule includes six buyer fields and five supplier fields as show in Table 1.

TABLE 1
Buyer Information

| | Buyer | Buyer | Buyer | | |
|---|---|---|---|---|---|
| | Submitted | Submitted | Submitted | Buyer | Buyer |
| Provider | Supplier | Item | Item Type | Submitted | Submitted |
| Division ID | Division ID | Number | Number | UOM | QOE |
| Memorial | Supplier | 1234-A | Manufacturer | Box | Null |
| Hospital | Division A | | Part Number | | |
| Supplier Information | | | | | |
| | | Supplier | | | |
| | Supplier | Item | Supplier Part | Supplier | Supplier |
| | Division ID | Number | Number Type | UOM | QOE |
| | Supplier | 1234-X | Manufacturer | BX | 5 |
| | Division B | | Part Number | | |

In one embodiment, IME rules can be provided to the CIS 116 via the IME interface, which can be run as a stand-alone feature. In another embodiment, the IME rules can be provided to the CIS 116 via a batch upload function, which allows data to be loaded through a batch file. The batch upload function can, for example, provide for input of the IME rules in the form of an Excel spreadsheet, an ASCII file, etc. In a related embodiment, a supplier 114 can only load IME rules that map to items in the electronic catalog.

In one embodiment, IME rules can be validated by mapping to valid items in the electronic catalog. Generally, if an IME rule is found for the line item (during IME processing steps 328, 332) and the IME rule maps to a valid item in the catalog, then the selected line item is validated. A supplier 114 may elect to have IME validation turned OFF or ON. If the supplier 114 elects to have IME validation turned OFF, then the IME rule will be applied and included in the processed purchase order (regardless of whether the rule maps to valid items in the catalog). If, however, the supplier 114 elects to have IME validation turned ON, then the IME rule will only be applied if the rule maps to valid items in the catalog. IME rules that do not map to valid items in the catalog will not be applied.

In step 336, the CIS 116 determines whether Refinery processing is ON for the trading partner relationship. If Refinery processing is OFF (No in step 336), the process proceeds to step 348. If Refinery processing is ON (Yes in step 336), the process continues to step 340.

In step 340, the line item is processed by the Refinery. The Refinery corrects product data in real time based on pre-established rules enabled by the supplier 114. Refinery rules can be applied to various fields, including part number, division ID, and UOM. Refinery rules can include rules that reformat the part number of the selected line item to a predetermined format acceptable to the supplier 114. For example, Refinery rules may be used to strip leading characters, trailing characters, or inner characters from a part number, strip specific characters within the part number, strip specific types of characters (such as non-alphanumeric, alphanumeric, numeric, alphabetic, and the like), insert leading characters or numbers, modify abbreviations or phrases, replace or modify the unit of measure, replace or modify company or department designations, determine company or department designations based on other fields submitted by the buyer (e.g., part number or UOM), and the like. Specific examples of Refinery rules can include: (1) leading division: if a part number has the following characters at the beginning (not including spaces at the beginning) xxxx, use yyyy as the division and strip those characters out of the part number; (2) trailing division: if a part number has the following characters at the end (not including spaces at the end) xxxx, use yyyy as the division and strip the characters out of the part number; (3) inner division: if a part number has the following characters contained within xxxx, use yyyy as the division and strip the characters out of the part number; (4) strip all characters: if xxxx is within the part number, remove from the part number; (5) strip non-alphanumeric: remove all non-alphanumeric string characters including spaces; (6) strip leading characters: strip the following leading characters (allow up to five) xxxx; (7) strip trailing characters: strip the following trailing characters (allow up to five) xxxx; (8) truncate leading characters: if the part number is more than x characters in length, strip the leading characters until the part number is that length in characters; (9) truncate trailing characters: if the part number is more than x characters in length, strip the trailing characters until the part number is that length in characters; (10) insert missing characters: if a part number is not x characters in length, add the following character y to the beginning of a part number until the part number is that many characters in length; (11) fill in leading characters: if xxxx is not in the yyyy position, insert that (those) character(s) before the position; (12) replace unit of measure: replace the following unit of measure xxxx with the following unit of measure yyyy. An example of a Refinery rule with reference to a trading partner relationship is “Remove all dashes from all item numbers coming from Memorial Hospital.”

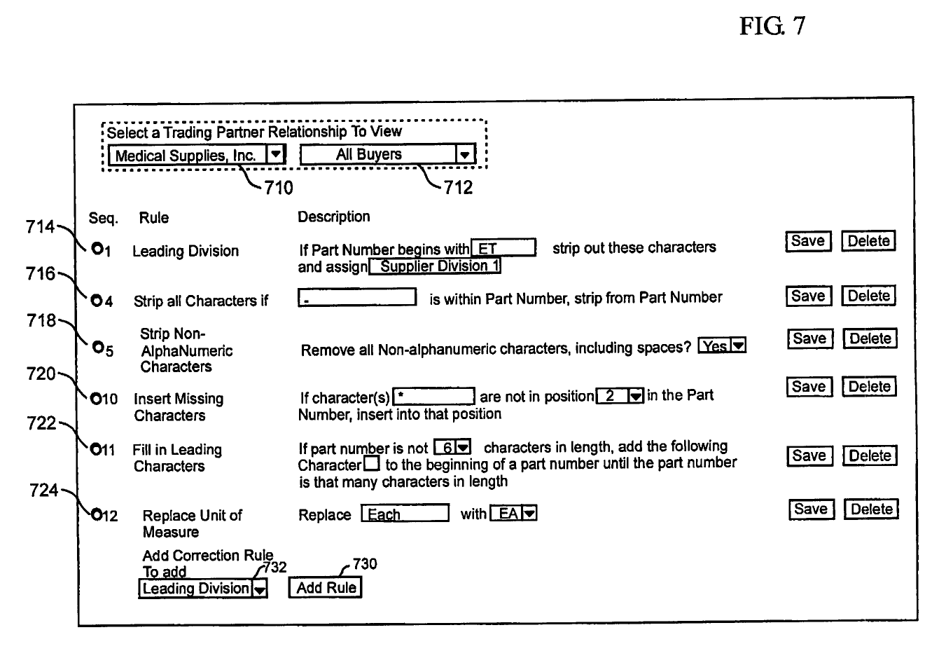

An example of a Refinery screen in accordance with one embodiment of the present invention is described below with reference to FIG. 7. Refinery rules can be set to apply to a particular buyer 110, a particular supplier 114, or a particular trading partner relationship. Alternatively, a Refinery rule can be set to apply to all buyers 110 associated with a supplier 114. In one embodiment, conflicting rules can be resolved by giving rules applicable to a specific buyer 110 precedent over rules applicable to all buyers 110. In one embodiment, Refinery rules can be provided to the CIS 116 via, for example, a browser based tool available to the supplier 114. Of course, other ways of entering Refinery rules are possible.

After the Refinery rules are applied to the selected line item (in step 340), processing proceeds to step 344, wherein a determination is made whether the selected line item has been corrected and whether the correction results in a valid item in the electronic catalog. If the line item has been corrected and the corrected line item is in the catalog (Yes in step 344), the corrected line item can be then be added to the processed purchase order in step 320.

Generally, if one or more Refinery rule(s) are applied and the application of the rule(s) results in a valid item in the catalog, then the selected line item is validated. In one embodiment, a supplier 114 may elect to have Refinery validation turned OFF. If so, the Refinery rule(s) will be applied regardless of the existence of a corresponding item in the catalog. The corrected line item can then be added to the processed purchase order in step 320.

If, in step 344, the line item has not been corrected, or application of the Refinery rule(s) does not result in a valid item in the electronic catalog (No in step 344), processing proceeds to step 348, wherein the line item is flagged and added to the processed purchase order (in step 320). In one embodiment, in the case where Refinery validation is ON and application of the Refinery rule(s) does not result in a valid item in the catalog, the original (uncorrected) line item is flagged (in step 348) and added to the processed purchase order (in step 320). Generally, a flagged line item on the processed purchase order indicates to the supplier 114 that the CIS 116 was unable to correctly correlate the flagged line item to a valid item (e.g., in the catalog).

In step 352, the process determines whether the purchase order contains additional line items that require processing. If so, (Yes in step 352), processing returns to step 308, wherein a new line item is selected to be processed. If the purchase order does not contain additional line items for processing (No in step 352), the process ends at step 356. The processed purchase order may thereafter be subjected to further processing (if it contains flagged items) or, alternatively, can be submitted to the supplier 114.

A flagged line item in a processed purchase order indicates that the CIS 116 was unable to validate the line item (using one or more of the processes described above with respect to FIG. 3). If a given purchase order contains errors, some suppliers 114 may want to make changes to the purchase order before it reaches their system, while other suppliers 114 may want to make changes to the purchase order once it has been received in their system.

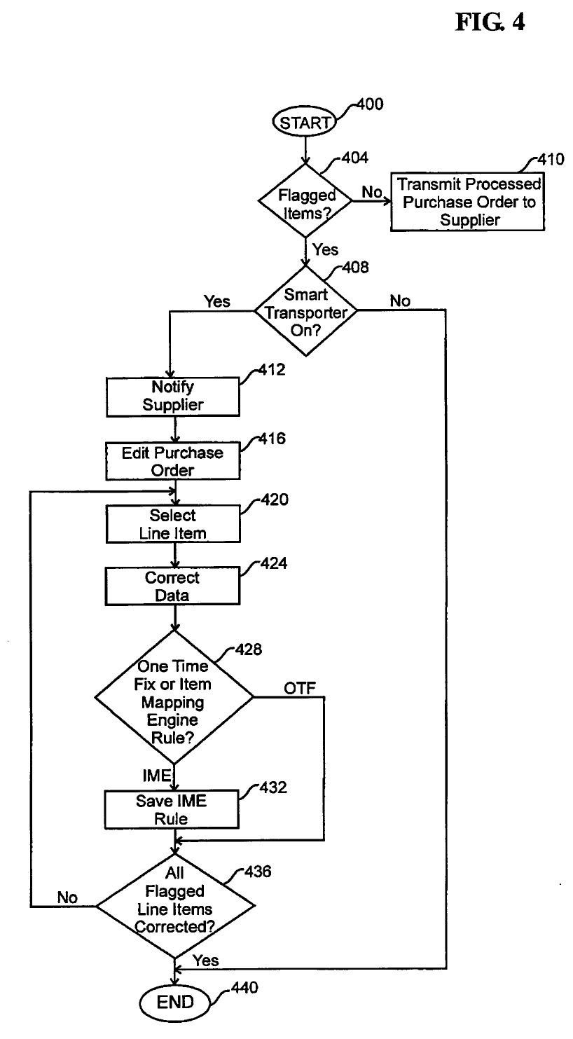

FIG. 4. is a flow diagram depicting a process, performed by a computer system, for further processing of purchase orders in accordance with one embodiment of the present invention. The process begins at step 400. In step 404, the CIS 116 determines whether the processed purchase order contains any flagged items. If so (Yes in step 404), the process continues to step 408. If there are no flagged items (No in step 404), then the line items in the processed purchase order are valid and the processed purchase order can be sent directly to the supplier 114 for fulfillment in step 410.

In step 408, the CIS 116 determines whether the supplier 114 wants to correct line items in the purchase order before it reaches their system (i.e., whether Smart Transporter processing is ON for the supplier 114 or the trading partner relationship). If so (Yes in step 408), processing continues to step 412. If not (No in step 408), processing ends at step 440.

In step 412, the supplier 114 is notified that a purchase order received by the CIS 116 contains at least one line item that could not be correlated to a corresponding product offered by the supplier 114. In one embodiment, the notification is sent to the supplier 114 via e-mail containing link to the specific purchase order within the CIS 116. By clicking on the link, the supplier 114 can be taken directly to the specific purchase order. In another embodiment, the notification can be by instant messaging or other types of computer communications. Alternatively, other types of notification may be used, such as a mailed letter, facsimile, phone call, voice mail, posting on a web site, and the like.

In an alternative embodiment, the supplier 114 is not alerted, but can periodically check for purchase orders that have been placed in suspension by the CIS 116. In this embodiment, the supplier 114 can access the CIS 116 and view a Purchase Order Index screen, which displays all purchase orders in suspension. The supplier 114 can then click on a suspended purchase order to view and work on that purchase order. In one embodiment, for each purchase order in suspension, the Purchase Order Index screen displays one or more of the following: (1) Buyer PO number, (2) Buyer Exchange ID, (3) Buyer Name, (4) Supplier Exchange ID, (5) Supplier Name, and (6) Suspended Date.

Upon receipt of the notification, the supplier 114 can (in step 416) enter the CIS 116 (e.g., via an internet browser or other interface) and edit the purchase order. The supplier 114 can access the purchase order on the CIS 116, for example, via a secure communications link over a public network. In one embodiment, line items containing errors (e.g., flagged line items) are highlighted such that the supplier 114 can quickly focus attention on the line items requiring correction. The CIS 116 thus allows the supplier 114 to examine and correct line items contained in the purchase order before the purchase order is sent to the supplier 114.

In step 420, the supplier 114 can select a flagged line item for correction. Then, in step 424, the supplier 114 can edit one or more fields of the line item, replacing incorrect data (e.g., part number) with correct data. In one embodiment, selection of a flagged line item brings up a separate edit line item screen wherein the supplier 114 can provide the correct supplier division, part number, part number type, QOE, UOM, etc. After each line item is corrected, any indication that the line item is invalid (such as highlighting) can be removed.

As the supplier 114 edits each line item, the supplier 114 can indicate whether the correction is to be applied as a “One Time Correction” (OTC) or whether the correction is to be saved for use with succeeding purchase orders (i.e., saved as an Item Mapping Engine Rule). In step 428, the process determines whether the supplier 114 has selected OTC or IME. If the supplier elects to have the correction saved as an IME rule for future use (IME in step 428), the process continues to step 432, wherein the correction is saved as an IME rule. In one embodiment, the supplier 114 has the option of selecting whether the new IME rule is to apply to all buyers 110 purchasing from the supplier 114, or whether the new IME rule is to apply only to the particular buyer 110 associated with this purchase order (i.e., this trading partner relationship). The process then proceeds to step 436. If, however, the supplier elects to have the correction be applied as a One Time Correction (OTC in step 428), the process proceeds directly to step 436.

In step 436, the supplier 114 determines whether all of the flagged line items have been corrected. If not all of the flagged line items have been corrected (No in step 436), the process returns to step 420 where the supplier 114 selects another line item for correction. If all of the flagged line items have been corrected (Yes in step 436), the process continues to step 440 wherein the process ends with a corrected purchase order. In one embodiment, once the supplier 114 has corrected all the flagged line items, the corrected purchase order can be re-submitted for validation by the CIS 116. Thus, the supplier 114 can receive into its system a purchase order containing valid line items. In an alternative embodiment, the corrected purchase order can be submitted directly to the supplier 114 without validation in the event that the supplier 114 wants to receive line items into their system that are not in the electronic catalog associated with the CIS 116.

In one embodiment, the corrected purchase order is electronically submitted to the supplier 114 via XML or EDI. Alternatively, the purchase order may be transmitted via other electronic or manual methods, such as facsimile, mail, courier, voice, and the like.

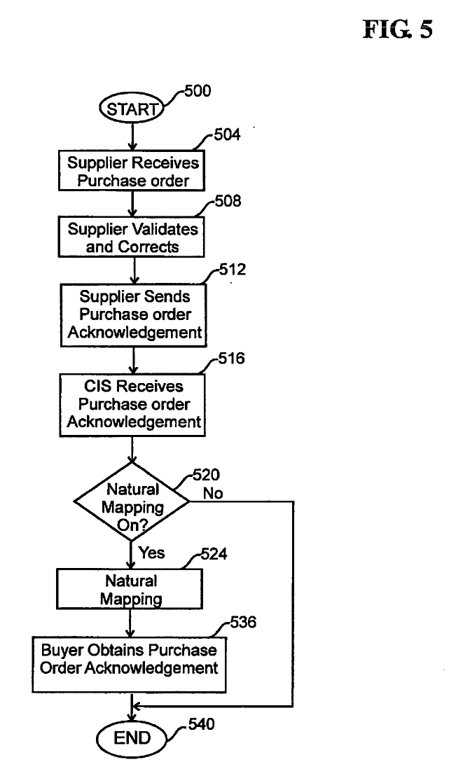

FIG. 5. is a flow diagram depicting a process, performed by a computer system, for processing of purchase orders in accordance with one embodiment of the present invention. The process begins at step 500. In step 504, the supplier 114 receives a purchase order from the network 112. In one embodiment, the purchase order received by the supplier 114 is the original (unprocessed) purchase order submitted by the buyer 110 and may contain invalid line items. In another embodiment, the purchase order has been processed by the CIS 116 and may contain line items that have been corrected by the CIS 116, along with invalid line items that the CIS 116 was unable to validate. In a related embodiment, the purchase order has been processed by the CIS 116 and may contain one or more flagged line items, indicating to the supplier 114 that the CIS 116 was unable to validate those particular line items.

While some suppliers 114 may want to edit and/or correct a purchase order before it reaches their system, other suppliers 114 will want to make changes to the purchase order once it has been received in their system. In step 508, the supplier 114 validates the line item(s) in the purchase order and corrects any invalid line item(s). In one embodiment, the supplier 114 verifies that the purchase order contains valid information, such as part number, UOM, QOE, etc. before attempting to correct the line items.

In step 512, the supplier 114 acknowledges receipt of the purchase order by sending a purchase order acknowledgement to the CIS 116. In one embodiment, the purchase order acknowledgement contains invalid line items (as determined by the supplier 114) and corresponding valid line items (as corrected). In another embodiment, the invalid line items are replaced by valid line items. In one embodiment, the purchase order acknowledgement is electronically transmitted to the CIS 116 (e.g., via XML or EDI). Alternatively, the purchase order acknowledgment may be transmitted via other electronic or manual methods, such as facsimile, mail, courier, voice, and the like.

In step 516, the CIS 116 receives the purchase order acknowledgement. Then, in step 520, the CIS 116 determines whether Natural Mapping processing is ON for the supplier 114 or the trading partner relationship. If so (Yes in step 520), processing continues to step 524. If not (No in step 520), processing ends at step 540.

In step 524, the CIS 116 performs natural mapping to update IME rules based on the changes to the line items that were made by the supplier 114. In one embodiment, IME rules are added or edited regardless of whether the line items were originally corrected (or flagged as invalid) by the CIS 116. For example, if a buyer 110 had specified a line item as containing part number “A123,” and part number “A123” exists in the electronic catalog, then the line item would not have been corrected (or determined to be invalid) by the CIS 116. If the supplier 114, however, had replaced part number “Al23” with a new model referred to by part number “B123,” natural mapping can be used to create an IME rule to replace part number “A123” with part number “B 123” on succeeding purchase orders. Natural mapping processing can advantageously identify differences between line items on the original purchase order and line items on the purchase order acknowledgement and update the IME rules used by the CIS 116.

In step 536, the buyer 110 obtains the purchase order acknowledgement (e.g., the purchase order acknowledgement is transmitted or made available to the buyer 110). The process then ends at step 540.

FIG. 6 illustrates an example of an IME interface screen in accordance with one embodiment of the present invention. A user, such as an administrator of the CIS 116, a representative of the supplier 114, or the like, can be provided with an IME interface for use in modifying, deleting, or creating rules to be applied by the IME.

The IME interface provides the user with a list of rules that can be edited or deleted, and also allows the user to add additional IME rules. The list of rules can be specific to a particular buyer 110, a particular supplier 114, or a particular trading partner relationship. Furthermore, the user may be provided with a mechanism to filter the IME rules such that only those rules matching a specified criteria are displayed.

In the illustrated embodiment of FIG. 6, pull downs 610 and 612 provide an optional mechanism for the user to restrict the display of rules to only those rules involving a specific supplier 114, a specific buyer 110, a specific supplier 114 and a specific buyer 110 (i.e., a trading partner relationship), or the like. In particular, pull down 610 allows the user to specify a specific supplier 114, causing only those rules involving the supplier 114 specified by the user to be displayed. Pull down 612 allows the user to specify a specific buyer 110, causing only those rules involving the buyer 110 specified by the user to be displayed. As indicated by the selected entry in pull down 612, the user is optionally provided a selection for displaying rules involving a specific supplier 114 and all buyers 110. Furthermore, a user can quickly locate all IME rules related to specific part number by entering the part number in text entry box 614.

In the embodiment illustrated in FIG. 6, the display of the each rule comprises two rows of five columns each. The first row, indicated by reference numeral 616 and labeled “Buyer Info (Map From),” identifies incorrect information related to a specific part as identified by the buyer 110. The second row, indicated by reference numeral 618 and labeled “Supplier Info (Map To),” identifies the correct information corresponding to the part identified in row 616. Accordingly, as the IME rules are applied, a line item containing a part number corresponding to that of row 616 can be replaced by the part number shown in row 618. Reference numerals 620-628 indicate fields where the user can specify corrections that are to be made with, for example, supplier name, buyer name, part number, part number type, quantity of eaches, and unit of measure, respectively. Furthermore, the CIS 116 can allow the user to edit and/or delete selected rules by clicking on button 630 and button 632, respectively. While the illustrated embodiment depicts certain fields, different fields may be desirable in different systems and are within the scope of the present invention.

FIG. 7 illustrates an example of a Refinery interface screen in accordance with one embodiment of the present invention. The Refinery interface screen may be utilized by a user, such as an administrator of the CIS 116, a representative of the supplier 114, or the like, to edit or create rules to be applied by the Refinery. A user can also add a new rule from this interface.

The Refinery interface provides the user a list of Refinery rules that can be edited or deleted, and also allows the user to enable new Refinery rules. The rules can be specific to a particular buyer 110, a particular supplier 114, or a particular trading partner relationship. In the illustrated embodiment, radio buttons 710 and 712 provide an optional mechanism for the user to select a trading relationship to be displayed. More specifically, radio buttons 710, 712 allows the user to specify a supplier 114 (or group of suppliers) and a buyer 110 (or group of buyers). As indicated by the selected entry in radio button 712, the user is optionally provided with a selection for displaying Refinery rules applicable to a specific supplier 114 and all buyers 110.

The embodiment illustrated in FIG. 7. shows examples of Refinery rules that may be available. The CIS 116 can allow the user to specify, for example, a procedure for modifying a line item on a purchase order. The first rule illustrated (indicated by reference numeral 714) is an example of a rule for stripping specific characters from the beginning of a part number and changing a division identifier. In this example, wherein the leading characters is a supplier division ID, the part number can be associated with the correct supplier division after the leading characters have been removed. The second rule illustrated (indicated by reference numeral 716) is an example of a rule for striping specific characters from anywhere in the part number. For example, a buyer 110 may submit a part number containing dashes interspersed therein. The rule can thus remove the dashes from the part number. The third rule illustrated (indicated by reference numeral 718) is an example of a rule for removing all non-alphanumeric characters from the part number. The fourth rule illustrated (indicated by reference numeral 720) is an example of a rule that allows a specific character to be inserted into a specific position in the part number. The fifth rule illustrated (indicated by reference numeral 722) is an example of a rule that allows a leading character to be inserted into the part number if the part number is not of a specific length. The sixth rule illustrated (indicated by reference numeral 724) is an example of a rule that allows a unit of measure in a part number to be replaced with the proper terminology used by the supplier 114. While the illustrated embodiment depicts certain exemplary rules, other rules are possible.

In the illustrated embodiment, button 730 allows a user to add a new IME rule. As illustrated by the selected entry in pulldown 732, the user is optionally provided with a selection for adding a rule to edit a part number to remove a Leading Division indicator and to assign the part number to a selected division of the supplier 114.

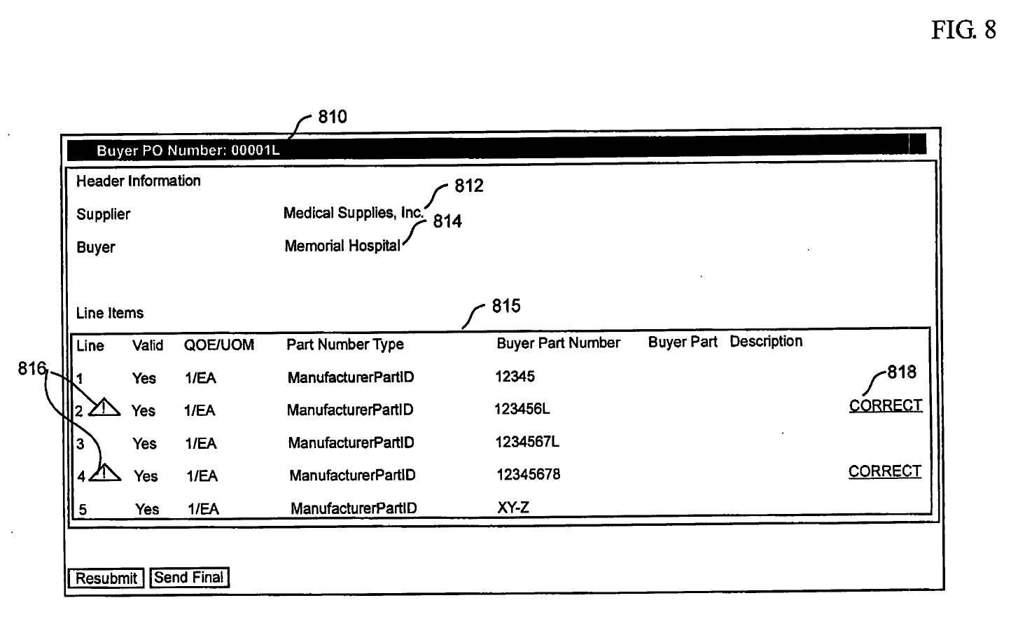

FIG. 8 illustrates an example of an interface screen that enables a supplier 114 to correct invalid line item(s) in a purchase order in accordance with one embodiment of the present invention. In the illustrated embodiment, reference numeral 810 indicates the purchase order number. Reference numerals 812 and 814 indicate the identity of the supplier 114 and the buyer 110, respectively.

In the illustrated embodiment, reference numeral 815 indicates an area in which each line item of the purchase order is listed. In one embodiment, all line items in the purchase order are listed. In an alternative embodiment, only flagged line items are listed. Reference numeral 816 indicates a caution sign or other indicia which points out the flagged line items (i.e., the particular line items that the CIS 116 was not able to validate). In addition to the caution sign, line items containing errors (e.g., flagged line items) can be highlighted (e.g., in yellow) such that the supplier 114 can easily identify on the line item(s) requiring correction. Other methods of identifying the flagged line items include shading, underling, coloring, flashing, background coloring, and the like.

In one embodiment, selection of a flagged line item (by clicking on the “Correct” button 818) brings up a separate edit line item screen wherein the supplier 114 can provide the correct part number, part number type, QOE, UOM, etc. In one embodiment, the supplier 114 is can indicate whether the correction is to be applied as a One Time Correction (e.g., by clicking on a “One Time Correction” button), or whether the correction is to be saved as an IME rule (e.g., by clicking on a “Save IME Rule” button). If the supplier 114 selects for the correction to be saved as an IME rule, the supplier 114 is given the option of selecting whether the new IME rule is to apply to only the particular buyer 110 who issued the instant purchase order, to a group of buyers 110 (including this particular buyer 110), or to all buyers 110. Thus, the supplier 114 can determine the specific trading partner relationship(s) to which the new IME rule applies.

Although the present invention has been fully described by way of examples and with reference to the accompanying drawings, it is to be understood that various changes and modifications will be apparent to those skilled in the art without departing from the spirit and scope of the invention. Therefore, unless such changes and modifications depart from the scope of the present invention, they should be construed as being included therein.

1. A method for updating a computer system for processing a purchase order from a buyer to a supplier, comprising the steps of:
comparing a purchase order from a buyer to a purchase order acknowledgement from a supplier to determine any changes to information in a line item of said purchase order made by said supplier, said changes including replacing said information with replacement information;
correlating said changes to develop a rule for replacing said information with said replacement information; and
adding said rule to a list of known errors that is specific to a trading partner relationship including said supplier and said buyer.

2. A method in accordance with claim 1, further comprising the steps of:
comparing original information in a line item to a catalog to determine whether said line item is valid; and
in the event that said line item is not valid,
accessing said list of known errors,
mapping said original information to said list of known errors, and
if said original information exists in said list of known errors, replacing said original information with corresponding replacement information from said list of known errors to derive a replacement line item.

3. A method in accordance with claim 1, wherein the step of comparing said purchase order includes the step of comparing information selected from the group consisting of:
supplier identifier;
supplier part number; and
supplier unit of measure.

4. A method in accordance with claim 1 wherein said list of known errors is specific to a trading partner relationship including said supplier and all buyers.

5. A computer-based system for updating the processing of a purchase order from a buyer to a supplier, comprising:
means for comparing a purchase order from a buyer to a purchase order acknowledgement from a supplier to determine any changes to information in a line item of said purchase order made by said supplier, said changes including replacing said information with replacement information;
means for correlating said changes and for developing a rule for replacing said information with said replacement information; and
means for adding said rule to a list of known errors that is specific to a trading partner relationship including said supplier and said buyer.

6. A computer-based system in accordance with claim 5, further comprising means, responsive to a determination that said line item is not valid, for:
(a) accessing said list of known errors,
(b) mapping said original information to said list of known errors, and
(c) if said original information exists in said list of known errors, for replacing said original information with corresponding replacement information from said list of known errors to derive a replacement line item.

7. A computer-based system in accordance with claim 5, wherein said means for comparing said purchase order is configured to compare information selected from the group consisting of:
supplier identifier;
supplier part number; and
supplier unit of measure.

8. A computer-based system in accordance with claim 5 wherein said means for adding said rule is configured to add said rule to a list of known errors that is specific to a trading partner relationship including said supplier and all buyers.

9. A method for updating a catalog of a buyer for use in ordering via an electronic purchase order from said buyer to a supplier, said method comprising the steps of:
receiving, from said buyer, a selection of a source of information;
receiving, from said buyer, a selection of product information from said source for inclusion in a file for updating said catalog, said selection of product information based on a review of items in said source of information;
generating, based on said selected product information, a file correlating said selected product information with corresponding replacement information from said source of information; and
providing said file for updating said selected product information in said catalog with said corresponding replacement information.

10. A method in accordance with claim 9 wherein said product information includes pricing information.

11. A method in accordance with claim 9, wherein said source of information contains discrepancy information created by comparing a purchase order from said buyer to a purchase order acknowledgement from said supplier to determine any changes to information in a line item of said purchase order, said changes including replacing said information with corresponding replacement information.

12. A method in accordance with claim 9, wherein said source of information contains discrepancy information created by comparing information in said catalog of said buyer to information in from a catalog of said supplier to determine any differences in said buyer information and said supplier information.

13. A method in accordance with claim 9, wherein said source of information is pricing information from a catalog associated with said supplier.

14. A method in accordance with claim 9, wherein said source of information is pricing information from a contract agreement associated with said supplier or a third party.

15. A method in accordance with claim 9, wherein said step of providing said file includes a step selected from the group consisting of:
automatically updating said catalog with said file;
transmitting said file to said buyer; and
providing said file for download by said buyer.

16. A method in accordance with claim 9, further comprising the steps of:
receiving, from said buyer, a selection of a file format for said file; and
converting said file into said selected file format.

17. A computer-based system for updating a catalog of a buyer for use in ordering via an electronic purchase order from said buyer to a supplier, comprising:
means for receiving, from said buyer, a selection of a source of information;
means for receiving, from said buyer, a selection of product information from said source for inclusion in a file for updating said catalog, said selection of product information based on a review of items in said source of information;
means for generating, based on said selected product information, a file correlating said selected product information with corresponding replacement information from said source of information; and
means for providing said file for updating said selected product information in said catalog with said corresponding replacement information.

18. A computer-based system in accordance with claim 17 wherein said product information includes pricing information.

19. A computer-based system in accordance with claim 17, wherein said source of information contains discrepancy information created by comparing a purchase order from said buyer to a purchase order acknowledgement from said supplier to determine any changes to information in a line item of said purchase order, said changes including replacing said information with corresponding replacement information.

20. A computer-based system in accordance with claim 17, wherein said source of information contains discrepancy information created by comparing information in said catalog of said buyer to information in from a catalog of said supplier to determine any differences in said buyer information and said supplier information.

21. A computer-based system in accordance with claim 17, wherein said source of information is pricing information from a catalog associated with said supplier.

22. A computer-based system in accordance with claim 17, wherein said source of information is pricing information from a contract agreement associated with said supplier or a third party.

23. A computer-based system in accordance with claim 17, wherein said means for providing said file selected from the group consisting of:
means for automatically updating said catalog with said file;
means for transmitting said file to said buyer; and
means for providing said file for download by said buyer.

24. A computer-based system in accordance with claim 17, further comprising:
means for receiving from said buyer a selection of a file format for said file; and
means for converting said file into said selected file format.

25. A method for processing a purchase order from a buyer to a supplier, comprising the steps of:
(a) selecting a line item in said purchase order for validation;
(b) comparing original information in said line item to a catalog containing items offered by said supplier to determine whether said line item is valid;
(c) in the event that said line item is not valid,
(1) accessing a rule specific to a trading partner relationship including said supplier and said buyer;
(2) refining said original information in accordance with said rule to derive replacement information for inclusion in a replacement line item;
(d) validating said replacement line item by comparing said replacement information to said catalog to determine whether said replacement line item is valid;
(1) including said replacement line item in a final purchase order to said supplier if said replacement line item is valid;
(2) including said line item containing said original information in said final purchase order if said replacement line item is not valid;
(e) transmitting said final purchase order to said supplier for processing;
(f) receiving a purchase order acknowledgement from said supplier;
(g) comparing said final purchase order to said purchase order acknowledgement to determine any changes to information in a line item of said purchase order made by said supplier, said changes including replacing said information with replacement information;
(h) correlating said changes to develop a rule for replacing said information with said replacement information; and
(i) adding said rule to a list of known errors that is specific to a trading partner relationship including said supplier and said buyer.

26. A method in accordance with claim 25, wherein the step of comparing original information includes the step of comparing original information selected from the group consisting of:
supplier identifier;
supplier part number; and
supplier unit of measure.

27. A method in accordance with claim 25, wherein the step of comparing original information includes the step of comparing a part number, and the step of accessing a rule includes a rule selected from the group consisting of:
a rule to remove a specific character from said part number;
a rule to remove a non-alphanumeric character from said part number;
a rule to insert a character in a position in said part number; and
a rule to fill in a leading character in said part number.

28. A method in accordance with claim 25, wherein the step of comparing original information includes the step of comparing a part number, and the step of accessing a rule includes a rule selected from the group consisting of:
a rule to truncate said part number if a number of characters in said part number exceeds a predetermined length;
a rule to insert a character in said part number until a number of characters in said part number is a predetermined length; and
a rule to replace a unit of measure with a different unit of measure.

29. A method in accordance with claim 25, wherein the step of comparing original information includes the step of comparing original information in said line item to a catalog that is specific to a trading partner relationship including said supplier and said buyer.

30. A method in accordance with claim 25, wherein the step of accessing a rule includes the step of accessing a rule that is specific to a trading partner relationship including said supplier and all buyers.

31. A method in accordance with claim 25, wherein the step of correlating said changes to develop a rule includes the step of developing a rule that is specific to a trading partner relationship including said supplier and all buyers.

32. A method in accordance with claim 25, further comprising the step of evaluating said purchase order to determine a trading partner relationship between said supplier and said buyer.

33. A computer-based system for processing a purchase order from a buyer to a supplier, comprising:
(a) means for selecting a line item in said purchase order for validation;
(b) means for comparing original information in said line item to a catalog containing items offered by said supplier and for determining whether said line item is valid;
(c) means, responsive to a determination that said line item is not valid, for
accessing a rule specific to a trading partner relationship including said supplier and said buyer; and
refining said original information in accordance with said rule to derive replacement information and for including said replacement information in a replacement line item;
(d) means for comparing said replacement line item to said catalog and for determining whether said replacement line item is valid;
(1) means, responsive to a determination that said replacement line item is valid, for including said replacement line item in a final purchase order to said supplier;
(2) means, responsive to a determination that said replacement line item is not valid, for including said line item containing said original information in said final purchase order;
(e) means for transmitting said final purchase order to said supplier for processing;
(f) means for receiving a purchase order acknowledgement from said supplier;
(g) means for comparing said final purchase order to said purchase order acknowledgement and for determining any changes to information in a line item of said purchase order made by said supplier, said changes including replacing said information with replacement information;
(h) means for correlating information in said discrepancy report and for developing a rule for replacing said information with said replacement information; and
(i) means for adding said rule to a list of known errors that is specific to a trading partner relationship including said supplier and said buyer.

34. A computer-based system in accordance with claim 33, wherein said means for comparing original information is configured to compare original information selected from the group consisting of:
supplier identifier;
supplier part number; and
supplier unit of measure.

35. A computer-based system in accordance with claim 33, wherein said means for comparing original information is configured to compare a part number, and said means for accessing a rule is configured to access a rule selected from the group consisting of:
a rule to remove a specific character from said part number;
a rule to remove a non-alphanumeric character from said part number;
a rule to insert a character in a position in said part number; and
a rule to fill in a leading character in said part number.

36. A computer-based system in accordance with claim 33, wherein said means for comparing original information is configured to compare a part number, and said means for accessing a rule is configured to access a rule selected from the group consisting of:
a rule to truncate said part number if a number of characters in said part number exceeds a predetermined length;
a rule to insert a character in said part number until a number of characters in said part number is a predetermined length; and
a rule to replace a unit of measure with a different unit of measure.

37. A computer-based system in accordance with claim 33, wherein said means for comparing original information is configured to compare original information in said line item to a catalog that is specific to a trading partner relationship including said supplier and said buyer.

38. A computer-based system in accordance with claim 33, wherein said means for accessing a rule is configured to access a rule that is specific to a trading partner relationship including said supplier and all buyers.

39. A computer-based system in accordance with claim 33, wherein said means for correlating said changes is configured to develop a rule that is specific to a trading partner relationship including said supplier and all buyers.

40. A computer-based system in accordance with claim 33, further comprising means for evaluating said purchase order to determine a trading partner relationship between said supplier and said buyer.

41. A method for processing a purchase order from a buyer to a supplier, comprising the steps of:
(a) selecting a line item in said purchase order for validation;
(b) comparing original information in said line item to a catalog containing items offered by said supplier to determine whether said line item is valid;
(c) in the event that said line item is not valid,
(1) accessing a rule specific to a trading partner relationship including said supplier and said buyer;
(2) refining said original information in accordance with said rule to derive replacement information for inclusion in a replacement line item;
(d) validating said replacement line item by comparing said replacement information to said catalog to determine whether said replacement line item is valid;
(e) in the event that said replacement line item is not valid,
(1) suspending said purchase order;
(2) notifying said supplier that said purchase order contains an invalid line item;
(3) allowing said supplier to access said purchase order to correct said invalid line item by replacing original information in said invalid line item with corresponding replacement information;
(4) including a replacement line item containing said replacement information in a final purchase order to said supplier;
(f) developing a rule for replacing said information with said replacement information;
(g) adding said rule to a list of known errors that is specific to a trading partner relationship including said supplier and said buyer;
(h) receiving, from said buyer, a selection of a source of information;
(i) receiving, from said buyer, a selection of product information from said source for inclusion in a file for updating said catalog, said selection of product information based on a review of items in said source of information;
(j) generating, based on said selected product information, a file correlating said selected product information with corresponding replacement information from said source of information;
(k) providing said file for updating said selected product information in said catalog with said corresponding replacement information.

42. A method in accordance with claim 41, further comprising the steps of:
(l) comparing original information in a line item to a catalog to determine whether said line item is valid; and
(m) in the event that said line item is not valid,
(1) accessing said list of known errors,
(2) mapping said original information to said list of known errors, and
(3) if said original information exists in said list of known errors, replacing said original information with corresponding replacement information from said list of known errors to derive a replacement line item;
(n) wherein said steps (l) and (m) are performed prior to said step (b).

43. A method in accordance with claim 41 wherein said product information includes pricing information.

44. A method in accordance with claim 41, wherein said source of information contains discrepancy information created by comparing a purchase order from said buyer to a purchase order acknowledgement from said supplier to determine any changes to information in a line item of said purchase order, said changes including replacing said information with corresponding replacement information.

45. A method in accordance with claim 41, wherein said source of information contains discrepancy information created by comparing information in said catalog of said buyer to information in from a catalog of said supplier to determine any differences in said buyer information and said supplier information.

46. A method in accordance with claim 41, wherein said source of information is pricing information from a catalog associated with said supplier.

47. A method in accordance with claim 41, wherein said source of information is pricing information from a contract agreement associated with said supplier or a third party.

48. A computer-based system for processing a purchase order from a buyer to a supplier, comprising:
(a) means for selecting a line item in said purchase order for validation;
(b) means for comparing original information in said line item to a catalog containing items offered by said supplier and for determining whether said line item is valid;
(c) means, responsive to a determination that said line item is not valid, for
accessing a rule specific to a trading partner relationship including said supplier and said buyer; and
refining said original information in accordance with said rule to derive replacement information for inclusion in a replacement line item;
(d) means for comparing said replacement line item to said catalog and for determining whether said replacement line item is valid;
(e) means, responsive to a determination that said replacement line item is not valid, for:
suspending said purchase order;
notifying said supplier that said purchase order contains an invalid line item;
allowing said supplier to access said purchase order to correct said invalid line item by replacing original information in said invalid line item with corresponding replacement information; and
including a replacement line item containing said replacement information in a final purchase order to said supplier;
(f) means for developing a rule for replacing said information with said replacement information;
(g) means for adding said rule to a list of known errors that is specific to a trading partner relationship including said supplier and said buyer;
(h) means for receiving, from said buyer, a selection of a source of information;
(i) means for receiving, from said buyer, a selection of product information from said source for inclusion in a file for updating said catalog, said selection of product information based on a review of items in said source of information;
(j) means for generating, based on said selected product information, a file correlating said selected product information with corresponding replacement information from said source of information;
(k) means for providing said file to said buyer for updating said selected product information in said catalog with said corresponding replacement information.

49. A computer-based system in accordance with claim 48 further comprising means, responsive to a determination that said replacement line item is not valid, for:
accessing said list of known error;
mapping said original information to said list of known errors, and
replacing said original information with corresponding replacement information from said list of known errors to derive a replacement line item, if said original information exists in said list of known errors.

50. A computer-based system in accordance with claim 48 wherein said product information includes pricing information.

51. A computer-based system in accordance with claim 48, wherein said source of information contains discrepancy information created by comparing a purchase order from said buyer to a purchase order acknowledgement from said supplier to determine any changes to information in a line item of said purchase order, said changes including replacing said information with corresponding replacement information.

52. A computer-based system in accordance with claim 48, wherein said source of information contains discrepancy information created by comparing information in said catalog of said buyer to information in from a catalog of said supplier to determine any differences in said buyer information and said supplier information.

53. A computer-based system in accordance with claim 48, wherein said source of information is pricing information from a catalog associated with said supplier.

54. A computer-based system in accordance with claim 48, wherein said source of information is pricing information from a contract agreement associated with said supplier or a third party.
